(19) United States
(12) Patent Application Publication
(10) Pub. No.: US 2006/0033880 A1
(43) Pub. Date: Feb. 16, 2006

(54) Method and apparatus for self-adjusting color scheme

(75) Inventor: Jose Eduardo Korneluk, Boynton Beach, FL (US)

Correspondence Address: MOTOROLA, INC;INTELLECTUAL PROPERTY SECTION, LAW DEPT, 8000 WEST SUNRISE BLVD, FT LAUDERDAL, FL, 33322, US

(73) Assignee: Motorola, Inc., Schaumburg, IL (US)

(21) Appl. No.: 10/898,843

(22) Filed: Jul. 26, 2004

(51) Int. Cl. A61B 3/02

(52) U.S. Cl. 351/242; 351/222

(57) ABSTRACT

A method (100) and apparatus (200) for configuring a display (205) to present data with a color contrast that enables perception of the data by a user who is colorblind. The method can include determining a colorblind condition of the user based on a user input. A color scheme then can be automatically selected for data presented on a display (205). The color scheme can be selected to define at least one color in which the data is displayed to enable perception of the data by the user.

### BACKGROUND OF THE INVENTION

1. Field of the Invention

The present invention generally relates to color displays, and more particularly to color displays that present a plurality of selectable display color schemes.

2. Description of the Related Art

Colorblindness affects 5% to 8% of men and 0.5% of women world-wide. There are various types of colorblindness and colorblindness also affects people in varying degrees. The most common broad category of colorblindness is often called red-green colorblindness, which includes the conditions of protanopia and protanomaly (red deficiencies), and deuteranopia and deuteranomaly (green deficiencies). People affected by red-green colorblindness generally have a hard time differentiating between the colors red and green. There is also some evidence that people with red-green colorblindness confuse yellows, oranges, and beiges with greens and reds.

Tritanopia is a form of colorblindness that is much less common than red-green colorblindness. Tritanopia is the insensitivity to shades of blue. People with tritanopia typically confuse blues and greens, but their perception of yellows is also affected in that shades of yellow seem to disappear or appear as lighter shades of red. An extremely small minority of people are affected by monochromacy. Those having monochromacy do not see colors, but see only different degrees of lightness. For them, the world appears to be shades of gray, black and white.

There are a variety of color deficiency tests currently available for identifying colorblindness in people. One such test is the pseudoisochromatic plate test. In this test, a series of test patterns, each comprising an arrangement of colored dots, is presented to a person. The type of colorblindness the person has is determined by which images the person can and can't see in the various patterns used for the test. The most common pseudoisochromatic plate test is the “Ishihara Test for Color Blindness.” The “Dvorine pseudoisochromatic plate tests” is another often used test.

A different type of colorblindness test uses a device called an “anomaloscope.” An anomaloscope is a device that tests for anomalies of color vision by displaying patterns of two or more colors that are to be matched to a reference color by the person being tested. For example, a “Nagel” anomaloscope presents a bipartite test pattern having an upper field and a lower field. In the upper field, red and green colors are displayed. The person adjusts a mixture of the red and green colors until the mixture matches the hue of a reference color, which in this case is yellow, presented in the lower field. The luminance of the reference color is then adjusted by the user until the color of the lower and upper fields look exactly the same to the user. Readings are then taken of the chosen color ratio and luminance. How much these readings differ from normal values provides an indication of the type of color deficiency. It should be noted that in addition to the Nagel anomaloscope, there are more sophisticated anomaloscopes that require mixing and matching a greater number of colors, but the premise of the testing procedure remains the same.

Although technological progress has provided a multitude of testing methods for identifying different types of colorblindness, it has also presented a number of obstacles to people who suffer from the disease. For example, displays for computers, mobile telephones and personal digital assistants (PDAs) were initially monochrome. Hence, the use of these devices did not present an obstacle for colorblind people. However, the technology of these devices has evolved so that the devices now are typically provided with color displays. To those who are colorblind, substantive data presented on the color displays is not always clearly distinguishable from background, graphics and other information that is also presented. Hence, the usefulness of these modern devices diminishes for those who are colorblind.

### SUMMARY OF THE INVENTION

The present invention relates to a method for configuring a display to present data with a color contrast that enables perception of the data by a user who is colorblind. The method can include determining a colorblind condition of the user based on a user input. A color scheme then can be automatically selected for data presented on a display. The color scheme can be selected to define at least one color in which the data is displayed to enable perception of the data by the user.

A menu of colorblind types can be displayed from which a user can select his type of colorblindness, if known. If the user does not know his type of colorblindness, one or more test patterns can be presented to the user. For each test pattern, the user can select at least one identifier corresponding to an image visually perceived by the user. The user's selection can be compared to predetermined data corresponding to the test pattern. The automatic color scheme selection can be based on a combination of the user's selections.

The present invention also relates to an apparatus for selecting a display color scheme. The apparatus can include a display, a user input device and a processor. The processor can be responsive to the user input device. The processor can determine a colorblind condition of a user based on at least one user input and automatically select a color scheme for data presented on the display. The color scheme can be selected to define at least one color in which the data is displayed to enable perception of the data by the user.

### BRIEF DESCRIPTION OF THE DRAWINGS

FIG. 1 is a flow chart showing a method for configuring a display in accordance with an embodiment of the present invention.

FIG. 2 is a diagram of an apparatus incorporating a display which is useful for understanding the present invention.

FIG. 3 is a block diagram of the apparatus of FIG. 2.

### DETAILED DESCRIPTION

An embodiment in accordance with the present invention relates to a method for automatically selecting a color scheme of a display to compensate for a colorblind condition of a user. More particularly, if a user suffers from a colorblind condition, the type of colorblindness the user suffers can be determined. The color scheme then can be selected to render display data with a color contrast that, for the colorblind user, distinguishes substantive data from background, graphics and other information presented on the display. For example, at least one color in which the substantive data is displayed can be defined to enable perception of the data by the user.

The type of colorblindness the user suffers can be determined in a number of ways. In one arrangement, a menu can be presented from which the user selects a colorblindness type. In another arrangement, the type of colorblindness can be determined by presenting one or more test patterns to the user and processing the user's responses to the test patterns.

Once the type of colorblindness is determined, an appropriate display color scheme then can be automatically selected. Alternatively, a menu comprising a plurality of display color schemes, each automatically selected to compensate for the user's colorblind condition, can be presented. The user then can select a display color scheme from the menu.

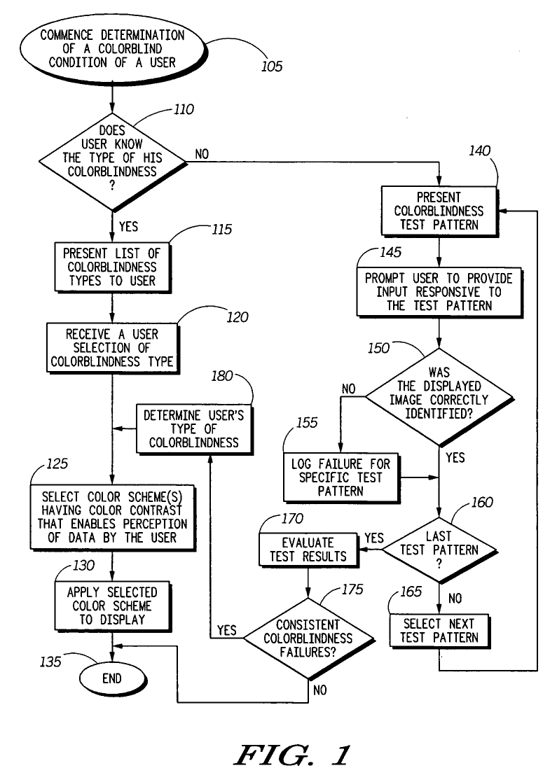

Referring to FIG. 1, a flowchart is shown which presents a method 100 for configuring a display in accordance with an embodiment of the present invention. Beginning at step 105, a device can commence determining a colorblind condition of a user based on at least one user input. For instance, the user can initiate a colorblindness compensation menu on the device. The device can be, for example, a mobile telephone, a personal digital assistant (PDA), a computer, a television, or any other device comprising a color display. The colorblindness compensation menu can be presented in a high contrast color scheme, for example black and white, so that the menu can be read by all users, regardless of each user's particular colorblind condition.

Proceeding to decision box 110, a user response can be solicited to determine whether the user knows the type of colorblindness he suffers from. For example, the user can be prompted to enter a response using a keypad, a stylus, a mouse, a touch screen, a remote control unit, or any other user input device. If the user does know his type of colorblindness, a list of colorblindness types can be presented to the user, as shown in step 115. A user input then can be received to select a colorblindness type from the menu, as shown in step 120.

Continuing to step 125, in response to the user input, the device can commence automatically selecting at least one color scheme to compensate for the user's type of colorblindness. The selected color scheme can be retrieved from a data store in the device or system wherein the color scheme is associated with the colorblindness type. For instance, a data table or text file wherein colorblindness types are associated with suitable color schemes can be queried. Referring to step 130, the selected color scheme then can be applied to the color display. For instance, the display's color palette can be adjusted in accordance with the selected color scheme. The process then can end until reinitiated, as shown in step 135.

In an alternate arrangement, a plurality of color schemes that compensate for the user's type of colorblindness can be identified in response to a user selection of a colorblindness type. The selected color schemes then can be presented to the user in a color scheme menu. A user response can be solicited to choose from the menu one of the color schemes to be applied to the color display.

Referring again to decision box 110, if the user does not know the type of colorblindness from which he suffers, a colorblindness test pattern can be presented to the user, as shown in step 140. For example, the colorblindness test pattern can be a test pattern of a pseudoisochromatic plate test. Examples of suitable test patterns are commercially available through a variety of sources, for instance Richmond Products, Inc. of Boca Raton, Fla. It should be noted, however, that the invention is not limited to any particular set of test patterns; any test pattern useful for identifying a type of colorblindness can be used.

Proceeding to step 145, the user can be prompted to enter an input responsive to the test pattern. For instance, the user can be prompted to identify an image perceived in the test pattern. For example, if the user perceives an alphanumeric character which has a correlating key in the device's keypad, the user can enter the alphanumeric character using the keypad. In another arrangement, the user can select a symbol correlating to the perceived image from a menu of symbols presented to the user on the display, for instance below the test pattern. The user also can be prompted to enter a particular input if the user is unable to identify the image in the test pattern.

Continuing to decision box 150 and step 155, if the user input does not correctly identify the displayed image, a failure can be logged for the specific test pattern. Referring to decision box 160 and step 165, if there are more test patterns to be presented to the user to complete the test, the next test pattern can be presented, as shown in step 140, and the image identification process can repeat.

After the last test pattern has been displayed and a correlating user input has been received, the user test results can be evaluated, as shown in step 170. Proceeding to decision box 175, if the logged failures are not consistent with failures that would be anticipated for a colorblind person, the color scheme selection process can be terminated. If, however, the logged failures are consistent with colorblindness, the user's type of colorblindness can be determined, as shown in step 180. For example, a data table or text file which correlates failure patterns to specific colorblindness types can be queried. After the type of colorblindness is determined, one or more color schemes can be automatically identified to compensate for the user's colorblind condition and a selected color scheme can be applied to the display, as previously described for steps 125 and 130. The process then can end, as shown in step 135.

In the arrangement described, colorblindness test patterns were selected from a pseudoisochromatic plate test. Nonetheless, the invention is not so limited. The test pattern can be any type of graphical pattern to which a person can be prompted to respond, and for which the person's response can be evaluated to determine whether the person suffers from colorblindness, and if so, identify the type of colorblindness. For example, the test pattern can be a pattern normally displayed by an anomaloscope, and correlating user inputs for matching colors can be received to determine a person's colorblind condition.

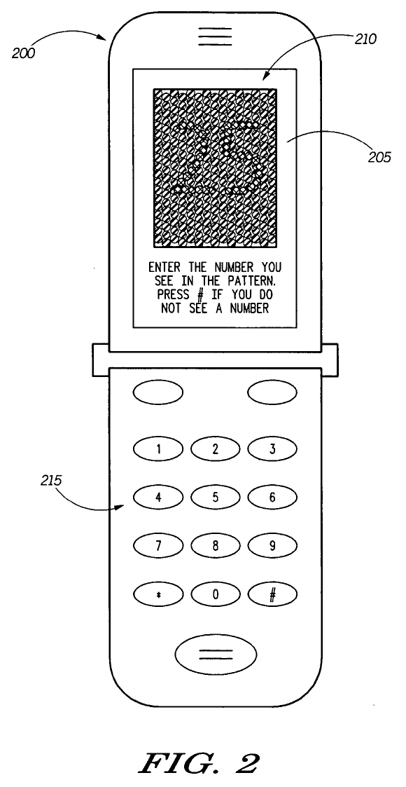

FIG. 2 is a diagram of an apparatus 200 that provides a plurality of selectable display color schemes to compensate for a colorblind condition of a user in accordance with the previously described method. The apparatus 200 is shown as a mobile telephone; however, the invention is not limited in this regard. As noted, the apparatus 200 can be a PDA, a computer, a television, or any other device having a color display 205 suitable for presenting a test pattern 210. In one arrangement, the color display 205 can be configured as a touch screen to receive user inputs, although the apparatus 200 also can include an input device 215 for such purpose. As shown, the user input device 215 is a keypad, but other input devices can be provided. For instance, the user input device 215 can be a mouse, a remote control unit, or any other user suitable input device.

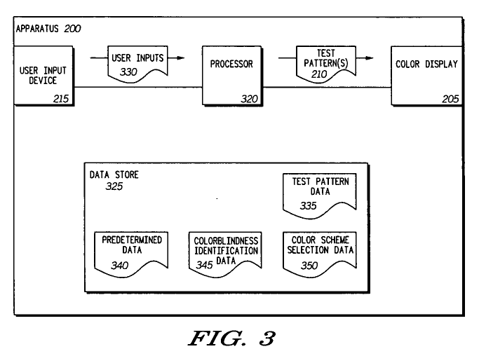

A block diagram of the apparatus 200 is shown in FIG. 3. In addition to the color display 205 and user input device 215, the apparatus 200 also can include a processor 320 and a data store 325. The processor 320 can be a central processing unit (CPU), a digital signal processor (DSP), an application specific integrated circuit (ASIC), or any other processor suitable for processing user inputs 330 and providing one or more test patterns 210.

The data store 325 can comprise an electronic storage medium, such as read only memory (ROM), flash memory or random access memory (RAM), a magnetic storage medium (e.g. a hard disk drive), an optical storage medium, a magneto-optical storage medium, or any other suitable data storage device. The data store 325 can store test pattern data 335 for generating the test pattern 210. The test pattern data 335 can be stored in an image format suitable to generate the test pattern 210. For example, the test pattern data 335 can be stored as a Joint Photographic Experts Group (JPEG) file, a Graphics Interchange Format (GIF) file, a Tag Image File Format (TIFF) file, a Portable Network Graphics (PNG) file or a bitmap (BMP) file. The image file can be generated by a graphics program or generated from a scanned image of a test pattern.

The data store also can store predetermined data 340 corresponding to images contained in the test pattern 210. For example, in the case that the test pattern 210 is selected from a pseudoisochromatic plate test, the predetermined data 340 can include alphanumeric characters or other information corresponding to one or more images contained in the test pattern 210. In the case that the test pattern 210 is selected from test patterns normally displayed by an anomaloscope, the predetermined data 340 can include luminosity and hue data previously discussed.

Additionally, the data store 325 also can store colorblindness identification data 345 for correlating user inputs to specific types of colorblindness, and color scheme selection data 350 for associating colorblindness types with color schemes that compensate for the colorblind conditions. The predetermined data 340, the colorblindness identification data 345, and the color scheme selection data 350 can be stored in data tables, text files, or any other format suitable for processing.

In operation, the processor 320 can propagate one or more of the test patterns 210 to the color display 205 for presentation. Test pattern data 335 for generating the test pattern 210 can be retrieved from the data store 325. The processor 320 also can receive the user inputs 330, such as those responsive to the presented test pattern 210, from the user input device 215. The processor 320 then can process the user inputs 330, for example by comparing user selections to the predetermined data 340 corresponding to the test pattern 210 that was presented. Based on the comparisons, the processor 320 can query the colorblindness identification data 345 to determine a type of colorblindness from which the user suffers. The processor 320 then can query the color scheme selection data 350 to automatically select at least one color scheme for the color display 205 which compensates for the user's colorblind condition.

The present invention can be realized in hardware, software, or a combination of hardware and software. This invention can be embodied in other forms without departing from the spirit or essential attributes thereof. While the foregoing is directed to the preferred embodiment of the present invention, other and further embodiments of the invention may be devised without departing from the basic scope thereof, and the scope thereof is determined by the claims that follow.

What is claimed is:

1. A method for configuring a display, comprising:
determining a colorblind condition of a user based on at least one user input; and
responsive to said determining step, automatically selecting at least one color scheme for the display which defines at least one color in which the data is displayed to enable perception of the data by the user.

2. The method according to claim 1, further comprising the step of displaying a menu of colorblindness types.

3. The method according to claim 2, further comprising the step of selecting the at least one user input to include a user selection of at least one colorblindness type from the menu.

4. The method according to claim 1, further comprising the step of presenting on the display at least one test pattern for determining a colorblindness type.

5. The method according to claim 4, further comprising the step of selecting the user input to include at least one user selection responsive to the test pattern.

6. The method according to claim 4, further comprising the step of selecting the user input to include a user selection of at least one identifier corresponding to an image visually perceived by the user in the test pattern.

7. The method according to claim 6, further comprising the step of comparing the user selection to predetermined data corresponding to the test pattern.

8. The method according to claim 1, further comprising the steps of:
presenting a plurality of test patterns to the user; and
selecting the at least one user input to include a plurality of user selections responsive to the plurality of test patterns.

9. The method according to claim 8, wherein said automatically selecting step further comprises the step of choosing the at least one color scheme for the display based on a combination of the plurality of user selections.

10. An apparatus for selecting a display color scheme, comprising:
a display;
a user input device; and
a processor responsive to said user input device, wherein said processor determines a colorblind condition of a user based on at least one user input and automatically selects at least one color scheme for data presented on said display, the color scheme defining at least one color in which the data is displayed to enable perception of the data by the user.

11. The apparatus of claim 10, wherein said processor selectively causes said display to present a menu of colorblindness types.

12. The apparatus of claim 11, wherein the user input corresponds to a user selection of at least one colorblindness type from the menu.

13. The apparatus of claim 10, wherein said display presents at least one test pattern to the user.

14. The apparatus of claim 13, wherein the user input corresponds to at least one user selection responsive to the test pattern.

15. The apparatus of claim 13, wherein the user input corresponds to a user selection of at least one identifier corresponding to an image visually perceived by the user in the test pattern.

16. The apparatus of claim 15, wherein said processor compares the user selection to predetermined data corresponding to the test pattern.

17. The apparatus of claim 10, wherein said display presents a plurality of the test patterns to the user, and the at least one user input corresponds to a plurality of user selections responsive to the plurality of test patterns.

18. The apparatus of claim 17, wherein said processor chooses the at least one color scheme for said display based on a combination of the plurality of user selections.

19. The apparatus of claim 10, wherein the apparatus is a communications device.

20. The apparatus of claim 10, wherein the apparatus is a mobile communications device.
